(19) United States
(12) Patent Application Publication
(10) Pub. No.: US 2003/0061666 A1
(43) Pub. Date: Apr. 3, 2003

(54) Leather processing

(75) Inventor: Tony Covington, Northampton (GB)

Correspondence Address: NOVOZYMES NORTH AMERICA, INC., 500 FIFTH AVENUE, SUITE 1600, NEW YORK, NY, 10110, US

(73) Assignee: BLC Leather Technology Centre Limited Leather Trade House, Northampton (GB)

(21) Appl. No.: 10/134,008

(22) Filed: Apr. 26, 2002

Related U.S. Application Data
(60) Provisional application No. 60/355,468, filed on Feb. 7, 2002.

(30) Foreign Application Priority Data
Dec. 4, 2001 (DK) 2001 01798
May 1, 2001 (GB) 0110695.4

(51) Int. Cl. C14C 11/00; C12P 21/06

(52) U.S. Cl. 8/94.2; 435/68.1

(57) ABSTRACT

The present invention relates to improvements in the processing of animal skins to create leather and results in improved leather quality, in terms of softness, and markedly increased area yield. According to the present invention there is provided a process for improving area yield and/or softness of leather which comprises treating chromium (III) or aldehyde tanned skins with an enzyme composition which is a mixture of at least one protease and at least one elastase. The invention applies particularly, although not exclusively, to clothing leather and upholstery leather production.

### CROSS-REFERENCE TO RELATED APPLICATIONS

This application claims, under 35 U.S.C. 119, priority of Danish application no. PA 2001 01798 filed on Dec. 4, 2001 and English Application no. 0110695.4 filed on May. 1, 2001, and this application claims the benefit of U.S. provisional application no. 60/355,468, filed on Feb. 7, 2001, the contents of which are fully incorporated herein by reference.

### FIELD OF THE INVENTION

This invention concerns improvements in the processing of animal skins to create leather. The invention results in improved leather quality, in terms of softness, and markedly increased area yield. The invention applies particularly, although not exclusively, to clothing leather and upholstery leather production.

### BACKGROUND OF THE INVENTION

The area of a piece of leather and, to a lesser extent, its softness are primarily controlled by structural features of the material from which the leather is made, that is hide or skin. This raw material comprises three main layers which each contribute to the properties of the piece.

The flesh layer is the part that was closest to the animal's body. It is composed of collagen fibres that have a distinctly low angle of weave, lying almost parallel to the surfaces of the hide or skin. This means that the layer has limited ability to stretch by distorting the weave horizontally and hence limits the area of the skin or leather.

The corium is the middle section and is the thickest part of the original skin. It is composed of a matrix of interconnecting collagen fibres: in the raw material the fibres have an average angle of weave close to 45°. The weave allows the skin or leather to adopt a larger area, if the angle is lowered by relaxation or straining, or to adopt a smaller area, if the angle of weave is raised during the leather making processes, for example by swelling the pelt.

The grain layer is the outermost part of the skin. It has a larger area than the corium and, because it is composed of very fine fibres, it is weaker than the corium, so it adopts a convoluted arrangement, which allows it to stretch without rupturing. The reversibility of the stretching mechanism is made possible by the presence of elastin, a fibrous protein which behaves very much like elastic.

The area of the skin or leather is determined by the corium angle of weave, which in turn is controlled by the area of the flesh layer, if it is present in the leather, and the area of the grain layer, which is usually present in the leather: of the two controlling mechanisms, the more important is the grain. In grain leather, the grain also limits the softness of the leather, since the presence of the elastin has a stiffening effect.

In order to increase area yield and softness of leather, it has been proposed to degrade the elastin. Applying elastolytic enzymes to raw, untanned hides or skins results in the desired area grain, but at the cost of (often unacceptable) looseness in the corium. The latter effect is due to the fact that elastolytic enzyme formulations or products have predominantly proteolytic activity and this causes considerable degradation to both the corium itself and to the non-structural proteins within the corium matrix, which contribute to the desired properties of the leather.

One solution to the problem would be to degrade the elastin by attacking it with elastase alone. However, there is no known source of elastase without accompanying protease. Furthermore, separating enzymes, to purify the elastase, is a high cost procedure, which makes the product prohibitively expensive for large scale industrial production.

### SUMMARY OF THE INVENTION

The present invention is based on the finding that mixtures of proteolytic and elastolytic enzymes can be successfully used to improve softness and area yield of leather by treating skins tanned with chromium (III) salts, or aldehydic tanning agents.

According to the present invention there is provided a process for improving area yield and/or softness of leather which comprises treating chromium (III) or aldehyde tanned skins with an enzyme composition which is a mixture of at least one protease and at least one elastase.

### DETAILED DESCRIPTION OF THE INVENTION

Enzyme mixtures containing protease(s) and elastase(s) are commercially available. They are typically derived from bacterial sources in the form of a so-called microbial protease, which in the absence of an expensive purification procedure also contains elastase. The present invention advantageously uses the relatively inexpensive unpurified “protease”. An example of en enzyme mixture commercially available is NovoCor® AX (available from Novozymes A/S).

The process of this invention exploits the difference in chemistry of the collagen and the elastin. Those differences are set out in Table 1, which contains some elements of their amino acid compositions: there is some dispute in the literature concerning the precise amino acid composition of these proteins, hence the figures quoted are indicative, based on published figures.

TABLE I
Indicative amino acid compositions of bovine type I collagen and
elastin (residues per 1000 residues).

| Amino acid type | Type I collagen | Elastin |
|---|---|---|
| Acidic (including amide) | 124 | 13 |
| Basic | 81 | 9 |
| Apolar | 316 | 570 |

It can be seen that elastin possesses an order of magnitude fewer acidic and basic groups on sidechains than collagen and almost double the amount of a polar side chains.

The basis of the invention is that by tanning using chromium (III) salts, chromium (III) is fixed to protein at the acidic side-chains, so the availability of such groups in collagen allows the tannage to work. Part of the definition of the tanning effect is that the protein acquires resistance to microbial attack i.e. putrefaction by the action of proteolytic enzymes. Hence, it has long been known that it is very difficult to modify the properties of chrome tanned leather by applying proteolytic enzymes, such as those used in the processes leading up to tanning. On the other hand, the lack of acidic groups in elastin means that chrome tanning has little effect on elastin, so there is no conferring of enzymatic resistance. Therefore, the elastin in chrome tanned pelt remains vulnerable to degradation by elastase. Consequently, treating chrome leather (leather tanned with chromium (III) salts) with an enzyme mixture containing an elastase and a protease will result in elastin degradation, but no damage to the collagen and other tanned non-structural proteins.

It is an important feature of the process of this invention that it is applied to chrome tanned leather, in which chromium (III) is covalently bound to the protein and hence is not displaced in solution; if it were displaced, the enzymes would be deactivated by the resulting tanning effect applied to themselves. This would occur if, for example, aluminium (III) or zirconium (IV) were to be included in the tannage. Typical chromium (III) tanning procedures are disclosed in Chem. Soc. Rev. 26(2), III, 1997 (Modern Tanning Chemistry—A.D. Covington).

The role of chromium (III) tannage also applies to covalent reaction at the amino groups i.e. by aldehydic tanning reactions, assuming the bound reagent is not released from a polymeric state by hydrolysis.

Suitable aldehydic tanning agents include aldehydes themselves, mono- and difunctional, aldehyde derivatives and compounds which have at least partial aldehydic function or reactive hydroxyl function, such as hydroxymethyl phosphonium salts, typically the sulphate or chloride, and especially oxazolidines. It is recognised that not all the potential crosslinkers are acceptable in the workplace because of toxicity hazards. In addition, all derivatives of glutaraldehyde produce leathers which are significantly coloured. Therefore, the preferred cross-linkers are the active-hydroxyl phosphonium salts, which are significantly less toxic than most of the other reagents and produce white leather.

Other tanning reactions that might be used prior to the treatment are likely to result in failure to gain a positive result; such tannages include vegetable tanning with plant polyphenols, syntan and resin tanning. The reason is that these reactions are labile, i.e. reversible, and they rely in some considerable part on forming hydrophobic interactions with the protein.

The process of the invention is particularly simple, merely requiring the enzymes to be added to the leather during the normal process of neutralisation prior to conventional post-tanning. Therefore, there is no extra process step involved in the overall treatment of skins to produce leather. This means that the process timing remains unaffected and, importantly, there is no capital cost associated with its introduction. This means that the new process can be applied in all tanneries.

The process is remarkably safe, with regard to damaging the leather. The pH of the leather does not have to be high, because the enzyme mixture can be used at a concentration high enough to produce the effect, without the necessity to operate at the pH optimum for the elastase. The resistance of the collagen is high, although it can be damaged, but not until extremely high concentration of protease is used at significantly elevated temperature, e.g. 50° C. Additional aspects of the safety of the process are: the reaction does not have to be prolonged for penetration by the enzyme, because access to the elastin is only a short distance through the grain surface and the elastin does not have to be completely dissolved, it is sufficient to cause significant degradation, so that its function is eliminated. The new technology has the advantage of not being restricted to specific relative activities of elastase and protease in the formulation.

The enzymatic reaction may preferably be carried out at a temperature in the range of 35 45θ C., more preferably around 40θ C., a pH preferably in the range of pH 5-8, more preferably pH 6-7, and a reaction time preferably in the range of 30-180 minutes, more preferably in the range of 60-120 minutes. The enzyme dosage may preferably be in the range of 2-10 kg enzyme product per ton of pelt, more preferably in the range of 3-5 kg enzyme product per ton of pelt. The enzyme product may have an activity measured in Löhlein Volhard Units (LVU) per gram in the range of 50,000 LVU/g to 250,000 LVU/g, preferably 100,000 LVU/g to 150,000 LVU/g.

One Löhlein-Volhard unit (LVU) is the amount of enzyme, which degrades 1.725 mg casein under the conditions set out here. Proteases degrade casein from an alkaline casein solution under the following standard conditions: Temperature 37θ C., pH 8.2 and reaction time 60 minutes. The reaction is stopped by adding HCl and non-degraded casein is precipitated with sodium sulphate. The filtrate's content of HCl which is not bound to degraded casein or its degradation products is determined by titration with NaOH. The more casein which is degraded and so non-precipitable, the more acid there will be in the filtrate. The consumption of NaOH in back titration therefore serves as a direct measure of the level of proteolytic activity.

It is a noteworthy feature of the invention, that the softness and area gains can be achieved without loosening the leather. This is due to two complementary factors. First, the relaxation of the corium is limited by the effects of the tannage, essentially fixing the fibre structure in place, so retaining much of the handle characteristics of the leather. Second, the resistance of the protein to proteolytic attack means that the non-structural protein is not removed nor is the collagen dissolved, so the filling of the fibre structure is maintained. Importantly, that resistance to degradation includes the grain-corium junction, where damage is seen as a loosening of the layers, resulting in poor break, i.e. coarse rippling of the grain surface when the leather is bent. The maintaining of the ‘tight’ structure is a vital quality determining factor in the finished leather.

The effectiveness of the invention is highlighted by treatment of ‘double face’ leather, e.g. English domestic wool sheepskins, following drying after chrome tanning. This is the worst case situation, because the flesh layer is still in place and the presence of the wool in the grain limits the ability of the grain to relax. Nevertheless, surprisingly it was found that the leather became significantly softer and measurably gained in area; see Example 1 below. In the case of chrome tanned upholstery leather the area gain can be considerable, up to 10%; see Example 2 below. The more powerful effect in the upholstery leather is because the pelt is split prior to tanning, so the tannage is applied only to the grain split and the restricting effect of the flesh layer on the ability of the grain and corium layers to relax is removed.

A major impact of the new technology lies in the increased profitability of the product. This is exemplified by a tannery processing about 50 tonnes of hide per day: the annual added profit from applying this invention would be about £3M.

The following three recipes give examples on the proposed use of the enzyme in the neutralization step.

Recipe 1:
UPHOLSTERY LEATHER WITH NOVOCOR AX
German bovine wetblue, 1.1-1.2 mm
All % refer to shaved weight

| Process | + | % | Product | ° C. | Time (min) | Notes |
|---|---|---|---|---|---|---|
| Neutralization | | 150 | Water | 40 | | |
| | | 2.0 | Sod.formate | | | |
| | | 2.0 | Tamol NA* | | 15 | |
| | + | 2.0 | Sod.bicarbonate | | 10 | |
| | + | 0.5 | Novocor AX | | 90 | PH 6.0-6.3 |

*Trade name of BASF,

Recipe 2:
DOUBLE FACE LAMBSKIN GARMENT WITH NOVOCOR AX
English domestic lambskin
Float ratio: 15 L/skin

| Process | + | G/L | Product | ° C. | Time (min) | Notes |
|---|---|---|---|---|---|---|
| Neutralization | | | Water | 35 | | |
| | | 2.0 | Sod.formate | | 20 | |
| | + | | | | | |
| | | 2.3 | Sod.bicarbonate | | 30 | PH 6.0-6.3 |
| | + | 0.5 | Novocor AX | | 90 | PH 6.0 |
| | + | 8.0 | Coripol MK* | | | |
| | | 2.0 | Propilon BNV/W* | | | |
| | | 1.5 | Borron SAF* | | 180 | |
| | + | 1.0 | Formic acid | | 180 | |

*Trade name of TFL

Recipe 3:
PHOLSTRY RETANNING WITH NOVOCOR AX
Raw material: Wet blue, Danish cows, 1.1-1.2 mm
All % refer to shaved weight:

| Process | + | % | Product | ° C. | Time (min) | Notes |
|---|---|---|---|---|---|---|
| Neutralization | | 110 | Water | 40 | | |
| | | 1.0 | Chromosal B* | | 60 | |
| | + | 2.0 | Sellasol NG** | | | |
| | | 2.0 | Sod.formate | | | |
| | | 0.5 | Novocor AX | | | |
| | + | 1.0-1.3 | Sod. bicarbonate | | 90 | PH 6.0-6.2 |

*Trade name of BASF,
**Trade name of TFL

The invention is further illustrated by the following non-limitative Examples.

### EXAMPLE 1

Wool sheepskins (50 pieces) in the dyed, crust state were wetted back, adjusted to pH 8.0 with sodium hydrogen carbonate, then treated with 1.0 wt-% Pyrase® 250MP (Trade Name for a proteolytic/elastolytic enzyme formulation supplied by Novozymes A/S) at 40° C. for 60 minutes. Pyrase® is a protease produced by surmerged fermentation of a genetically modified Bacillus.

After dyeing in the normal way, it was found that the softness had increased, markedly improving the handle. Area Measurement revealed that the average area gain of the experimental leathers was 3% greater than normal production. In this production, although the area gain is commercially important, the more significant result is the improvement in quality with regard to softness.

### Example 2

In two separate processes conducted in a tannery, single bovine upholstery hides, previously split in the limed state and chrome tanned all as usual, were neutralised to pH 7.0, when they were treated with 1.0 wt-% Pyrase® 250MP for 2 hours at 40° C.

TABLE II
Mean results for trials on upholstery hides.

| Treatment | Wet blue area (m²) | Crust area (m²) | Increase (%) |
|---|---|---|---|
| Control | 5.13 | 5.69 | 10.9 |
| Control | 5.90 | 6.53 | 10.7 |
| Pyrase | 5.02 | 5.98 | 19.1 |
| Pyrase | 5.32 | 6.41 | 20.4 |

From Table II, after drying in the normal way, the experimental hides were on average 9.0% bigger in area than untreated control hides, comparing the crust area with the wet blue area. In addition, the Pyrase treated hides were almost twice as strong, as measured by both tear and tensile strength.

1. A process for improving softness and/or area yield of leather, comprising
(a) providing animal skins tanned with chromium (III) salts or an aldehydic tanning agent, and
(b) treating the tanned skins with an enzyme mixture comprising a protease and an elastase.

2. The process of claim 1, wherein the enzyme mixture is a microbial protease with an elastase component.

3. The process of claim 1, wherein the enzyme mixture is added to the neutralisation bath preceding a post-tanning treatment.

4. The process of claim 1, wherein the enzymatic treatment is carried out at a temperature in the range of 35-45 θ C.

5. The process of claim 5, wherein the enzymatic treatment is carried out at a pH in the range 0f 6-7.

6. The process of claim 1, wherein the enzymatic treatment is carried out at a reaction time in the range of 30-180 minutes.

7. The process of claim 1, wherein the enzyme dosage is in the range of 2-10 kg enzyme product per ton of pelt.

8. The process of claim 1, substantially as described herein in Example 1 or 2.
